(19) United States
(12) Patent Application Publication
(10) Pub. No.: US 2007/0055081 A1
(43) Pub. Date: Mar. 8, 2007

(54) Processes for production of alpha-aminooxyketones and alpha-hydroxyketones

(76) Inventor: Susumu Saito, Nagoya-shi (JP)

Correspondence Address: FROMMER LAWRENCE & HAUG, 745 FIFTH AVENUE- 10TH FL., NEW YORK, NY, 10151, US

(21) Appl. No.: 11/506,573

(22) Filed: Aug. 18, 2006

Related U.S. Application Data
(63) Continuation-in-part of application No. PCT/JP05/02163, filed on Feb. 14, 2005.

(30) Foreign Application Priority Data
Feb. 20, 2004 (JP) 2004-044540

(51) Int. Cl. C07C 249/02; C07D 403/02

(52) U.S. Cl. 564/253; 548/254

(57) ABSTRACT

The present invention provides a method for easily obtaining α-aminooxyketone compound which is a synthetic equivalent for monosaccharide and pentoses, and a equivalent of α-hydroxyketone compound that can be synthetic intermediates of various physiologically active materials, in high yield; to pave the way for the synthesis of monosaccharide and furthermore of oligosaccharide from the resulting α-hydroxyketone compound induced from α-aminooxyketone compound; and to open new possibilities for the synthesis of various sugar medicines such as anticancer agents, antithrombogenic agents, anti-viral agents, anti-HIV agents, inhibitors of cholesterol synthesis, verotoxin neutralizing agents. According to the invention, a carbonyl compound is allowed to react with a nitroso compound to produce an α-aminooxyketone compound using a catalyst containing a heterocyclic compound shown in the general formula (I) (wherein X1, X2 and X3 independently represent nitrogen, carbon, oxygen or sulfur; and Z represents a substituted or unsubstituted 5- to 10-membered ring).

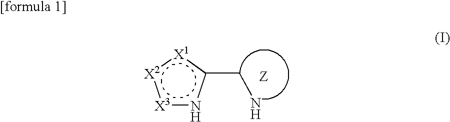

### INCORPORATION BY REFERENCE

This application is a continuation-in-part application of international patent application Serial No. PCT/JP2005/002163 filed Feb. 14, 2005, which claims priority to Japanese patent application Serial No. JP 2004-044540 filed Feb. 20, 2004.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates to a medicine, an organic material having physiological activity, α-aminooxyketone compounds which are materials of liquid crystal and the like, and a process for producing α-hydroxyketone compounds induced therefrom; as to a catalyst used for producing α-aminooxyketone compounds, specifically α-aminooxyketone compounds which can give optical isomers with high purity, and a process for producing α-hydroxyketone compounds induced therefrom, and a catalyst used for producing α-aminooxyketone compounds.

### BACKGROUND OF THE INVENTION

α-hydroxycarbonyl compounds are frequently found in natural organic compounds and the molecule skeletons of pharmaceuticals. They are synthetic equivalents for monosaccharides and pentoses, and are very important synthetic building blocks which can lead to various physiologically active materials, intermediates in the synthesis of medicines and liquid crystalline materials. The α-hydroxycarbonyl compounds easily lead to synthesis of optical isomers with high purity by asymmetric oxygenation of carbonyl compounds. However, asymmetric oxygenation at the α-position of the carbonyl group by the usual methods requires a two-step process: first, synthesizing and isolating enolate intermediates from carbonyl compounds, and second, performing with the use of a relatively expensive oxygen-introducing reagent; therefore, the asymmetric oxygenation has problems of low atom efficiency and the like.

In contrast, as a method for asymmetric oxygenation of ketones, methods for directly obtaining asymmetric oxygenation products of ketones without synthesizing and isolating enolate intermediates have been reported. The asymmetric oxygenation is to obtain α-aminooxyketones using amino acid, proline as a catalyst and nitrosobenzene as an oxygen-introducing reagent (see e.g. non-patent documents 1-3). However, many problems have thus far not been resolved in these systems, including low catalytic efficiency (10 to 20 mol % catalyst are needed), poor reproducibility and the like. Moreover, it is known that double oxygenation by nitrosobenzene progresses a side reaction.

Alternatively, it has been reported that α-aminooxyketone can be obtained in high yield from an alkylsilyl enol ether and nitrosobenzene with alkylsilyl triflate as a Lewis Acid catalyst (see e.g. Nonpatent document 4) and also from an alkyltin enolate and nitrosobenzene with Ag-BINAP complex as a catalyst (see e.g. Nonpatent document 5). Additionally, other methods have been disclosed to produce aldol products from the condensation reaction of carbonyl compounds: using a compound which has an ether and alcohol units within a certain molecule, in liquid CO₂ or supercritical CO₂, in the presence of an acid catalyst (see e.g. Patent document 1); performing the reaction in water, in the presence of boron acid, a surfactant or a Brönsted acid (see e.g. Patent document 2); using a lanthanide trifluoromethanesulfonate and a chiral crown ether as a catalyst (see e.g. Patent document 3); and the like.
Patent document 1: Japanese Laid-Open Patent Application No. 2002-284729
Patent document 2: Japanese Laid-Open Patent Application No. 2002-275120
Patent document 3: Japanese Laid-Open Patent Application No. 2002-200428
Nonpatent document 1: Brown, S. P., Brochu, M. P., Sinz, C. J., & MacMillan, D. W. C. (2003) J. Am. Chem. Soc. 125, 10808-10809
Nonpatent document 2: Zhong, G. (2003) Angew. Chem. Int. Ed. 42, 4247-4250
Nonpatent document 3: Hayashi, Y., Yamaguchi, J., Hibino, K., & Shoji, M. (2003) Tetrahedron Lett. 44, 8293-8296
Nonpatent document 4: Momiyama, N., Yamamoto, H. (2002) Angew. Chem. Int. Ed. 41, 2986-2987
Nonpatent document 5: Momiyama, N., Yamamoto, H. (2003) J. Am. Chem. Soc. 125, 60 38-6039

Citation or identification of any document in this application is not an admission that such document is available as prior art to the present invention.

### SUMMARY OF THE INVENTION

The object of the present invention is to provide a method for easily obtaining α-aminooxyketone compound which is a synthetic equivalent for monosaccharide and pentoses, and an equivalent of α-hydroxyketone compound that can be a synthetic intermediate of various physiologically active materials, in high yield; to pave the way for the synthesis of monosaccharide and furthermore of oligosaccharide from the resulting α-hydroxyketone compound induced from α-aminooxyketone compound; and to open new possibilities for the synthesis of various sugar medicines such as anticancer agents, antithrombogenic agents, anti-viral agents, anti-HIV agents, inhibitors of cholesterol synthesis, verotoxin neutralizing agents.

The applicant has shown, as displayed in the following formula, in an asymmetric nitroso aldol reaction between 3.0 equivalents of cyclohexanone (2a) and 1.0 equivalent of nitrosobenzene (3) using pyrrolidine tetrazole type catalyst (1) (5 mol %) induced from proline, in dimethyl sulfoxide (DMSO) at room temperature 25-30° C., the reaction was almost completed in only one hour, and the desired product (4a) was obtained in 94% chemical yield and >99% ee optical purity.

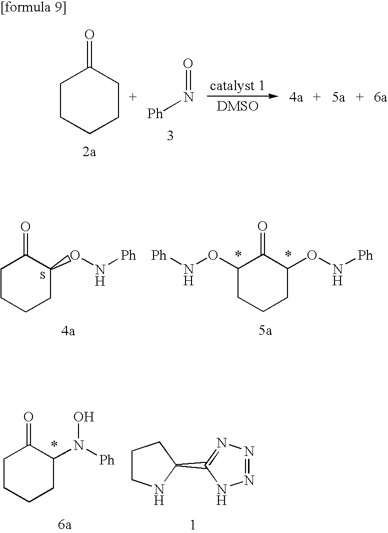

When proline was used as a catalyst under the exactly equal reaction condition, chemical yield stayed at 35% and at the same time almost 2% of the production of double additional product (5a) was identified. Even when the amount of catalyst to be used (1) was converted from 5 mol % to 3 mol %, and to 2 mol %, the reactions were progressed to observe a little decrease of the chemical yield to 72% and 50% respectively, but the optical purities were very high at >99% ee. Further, various carbonyl compounds were subjected to reaction, although reaction conditions should be adjusted slightly for aldehydes, high optical purities were obtained in any of the examples. The present invention has been thus completed based on this knowledge.

That is, the present invention relates to:
a process for producing an α-aminooxyketone compound wherein a carbonyl compound is reacted with a nitroso compound by using a catalyst containing a heterocyclic compound shown in the general formula (I) (wherein: X1, X2 and X3 independently represent nitrogen, carbon, oxygen or sulfur; Z represents a substituted or unsubstituted 5- to 10-membered ring) (“1”); 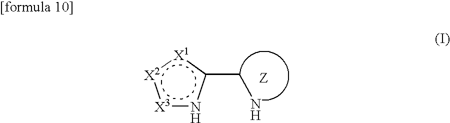
   preferably relates to:
the process for producing an α-aminooxyketone compound according to “1”, wherein the heterocyclic compound is a tetrazole derivative shown in the general formula (II) (wherein Z represents a substituted or unsubstituted 5- to 10-membered ring) (“2”); 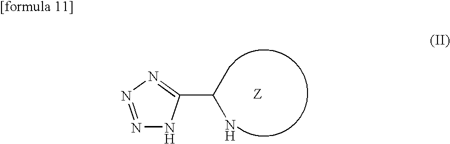
the process for producing an α-aminooxyketone compound according to “2”, wherein the tetrazole, derivative is a compound shown in the general formula (III) (“3”); 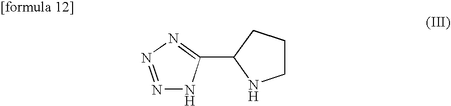
the process for producing an α-aminooxyketone compound according to any one of “1” to “3”, wherein the nitroso compound is nitrosobenzene (“4”);
the process for producing an α-aminooxyketone compound according to any one of “1” to “4”, wherein the carbonyl compound is a compound shown in the general formula (IV) (wherein R1 and R2 independently represent hydrogen, or a substituted or unsubstituted alkyl group, a substituted or unsubstituted alkenyl group, a substituted or unsubstituted alkoxy group, a substituted or unsubstituted amide group, or a substituted or unsubstituted aryl group; and R1 and R2 may bind to each other to form a ring) (“5”); 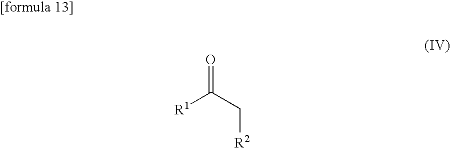
the process for producing an α-aminooxyketone compound according to “5”, wherein the α-aminooxyketone compound contains R asymmetric carbon, where R1 represents hydrogen in the general formula (IV) (“6”); the process for producing an α-aminooxyketone compound according to “5”, wherein the α-aminooxyketone compound contains S asymmetric carbon; where R1 represents a substituted or unsubstituted alkyl group, a substituted or unsubstituted alkenyl group, a substituted or unsubstituted alkoxy group, a substituted or unsubstituted amide group, or a substituted or unsubstituted aryl group, and either one of these groups bind to R2 to form a ring in the general formula (IV) (“7”); the process for producing an α-aminooxyketone compound according to any one of “1” to “7”, wherein a solvent containing dimethyl sulfoxide or methyl nitrile, or both of them is used (“8”); the process for producing an α-aminooxyketone compound according to any one of “1” to “8”, wherein reaction is performed at room temperature (20-30° C.) (“9”); and a process for producing an α-hydroxyketone compound wherein an α-aminooxyketone compound obtained from the process according to any one of “1” to “9” is reacted in a solvent by using CuSO₄ as a catalyst (“10”).

The present invention also relates to:
a catalyst for producing an α-aminooxyketone compound by which a carbonyl compound is reacted with a nitroso compound, wherein the catalyst contains a heterocyclic compound shown in the general formula (I) (wherein: X1, X2 and X3 independently represent nitrogen, carbon, oxygen or sulfur; Z represents a substituted or unsubstituted 5- to 10-membered ring) (“11”); 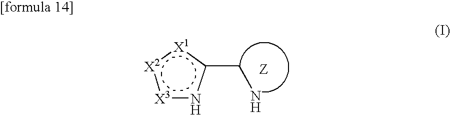
   preferably relates to:
the catalyst for producing an α-aminooxyketone compound according to “11”, wherein the heterocyclic compound is a tetrazole derivative shown in the general formula (II) (wherein Z represents a substituted or unsubstituted 5- to 10-membered ring) (“12”), 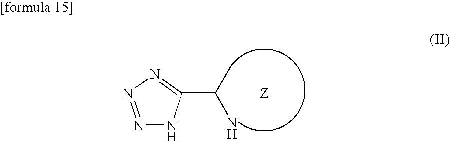
the catalyst for producing an α-aminooxyketone compound according to “12”, wherein the tetrazole derivative is a compound shown in the general formula (III) (“13”) 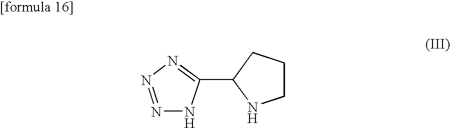
the catalyst for producing an α-aminooxyketone compound according to any one of claims “11” to “13”, wherein the nitroso compound is nitrosobenzene (“14”),
the catalyst for producing an α-aminooxyketone compound according to any one of “11” to “14”, wherein the carbonyl compound is a compound shown in the general formula (IV) (wherein R1 and R2 independently represent hydrogen, or a substituted or unsubstituted alkyl group, a substituted or unsubstituted alkenyl group, a substituted or unsubstituted alkoxy group, a substituted or unsubstituted amide group, or a substituted or unsubstituted aryl group; and R1 and R2 may bind to each other to form a ring) (“15”), 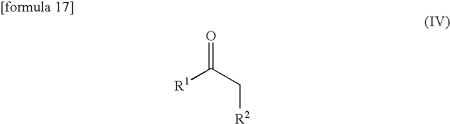
the catalyst for producing an α-aminooxyketone compound according to “15”, wherein the α-aminooxyketone compound contains R asymmetric carbon, where R1 represents hydrogen in the general formula (IV) (“16”), the catalyst for producing an α-aminooxyketone compound according to “15”, wherein the α-aminooxyketone compound contains S asymmetric carbon, where R1 represents a substituted or unsubstituted alkyl group, a substituted or unsubstituted alkenyl group, a substituted or unsubstituted alkoxy group, a substituted or unsubstituted amide group, or a substituted or unsubstituted aryl group, or where either one of these groups bind to R2 to form a ring in the general formula (IV) (“17”).

By using heterocyclic compound shown in the general formula (I) as a catalyst, without isolating intermediates, at a stage in an asymmetric oxygenation of carbonyl compounds, the amount of catalyst was reduced, the efficiencies of catalyst, atom and the like were increased, and consequently α-aminooxyketone compounds can be obtained in high chemical yield and high optical purity. The present invention paves the way for artificially and freely producing oligosaccharides, hexose and pentose skeletons which can be found in unit structures in DNA and RNA; and gives possibilities to develop various sugar medicines such as anticancer agents, antithrombogenic agents, anti-viral agents, anti-HIV agents, inhibitors of cholesterol synthesis, and verotoxin neutralizing agents.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DETAILED DESCRIPTION

A process for producing α-aminooxyketone compounds of the present invention is a process by using so-called nitroso aldol reaction which produces α-aminooxyketone compounds by an asymmetric oxygenation reaction of carbonyl compounds and nitroso compounds.

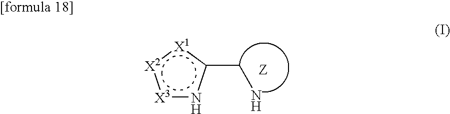

A process for producing α-aminooxyketone compound of the present invention is not especially limited as long as it is a process for producing an α-aminooxyketone compound wherein a carbonyl compound is reacted with a nitroso compound by using a catalyst containing a heterocyclic compound shown in the general formula (I) (wherein: X1, X2 and X3 independently represent nitrogen, carbon, oxygen or sulfur; Z represents a substituted or unsubstituted 5- to 10-membered ring).

A carbonyl compound used for a process for producing α-aminooxyketone compounds of the present invention is not especially limited as long as it has a carbonyl group, and may be a compound in a soluble form or a compound easily forming hydrates. Specifically, aldehyde compounds and ketone compounds, preferably carbonyl compounds shown in the general formula (IV) can be exemplified.

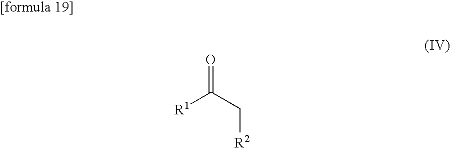

In the general formula (IV), R1 and R2 independently represent hydrogen, or a substituted or unsubstituted alkyl group, a substituted or unsubstituted alkenyl group, a substituted or unsubstituted alkoxy group, a substituted or unsubstituted amide group, or a substituted or unsubstituted aryl group; and R1 and R2 may bind to each other to form a ring. As an alkyl group represented by R1, R2 in the general formula (IV), it may be linear or cyclic, and alkyl groups with 1-30 carbons such as methyl, ethyl, n-propyl, n-butyl, n-pentyl, n-hexyl, n-heptyl, n-octyl, n-nonyl and n-decyl, and such as cyclobutyl, cyclopentyl, cyclohexyl, cycloheptyl, cyclooctyl, cyclononyl and cyclodecyl can be exemplified. As an alkenyl group represented by R1, R2 in the general formula (IV), alkenyl groups with 1-30 carbons such as vinyl, 1-propenyl, allyl, 1-butenyl, 2-butenyl, 1-pentenyl, and 1-hexynyl can be exemplified.

Further, as an alkoxy group, an alkoxycarbonyl group and an aryl group represented by R1, R2 in the general formula (IV), alkoxy groups with 1-30 carbons such as methoxy, ethoxy, n-propoxy, n-butoxy, n-pentyloxy, n-hexyloxy, cyclohexyloxy and phenyloxy; alkoxycarbonyl group with 1-30 carbons such as methoxy carbonyl, ethoxy carbonyl, butoxy carbonyl and pentyloxy carbonyl; and aryl groups with 1-30 carbons such as phenyl, 1-naphtyl, 2-naphthyl, 1-anthryl, 2-anthryl, 9-anthryl, 1-phenanthryl, 2-phenanthryl, benzyl and phenethyl; can be exemplified respectively.

As a substituent of alkyl group, alkenyl group, alkoxy group, alkoxy-carbonyl group and aryl group represented by R1, R2 in the general formula (IV), alkyl groups such as methyl, ethyl, n-propyl, n-butyl, cyclopentyl, cyclohexyl and cycloheptyl; alkenyl groups such as vinyl, 1-propenyl, allyl, and 1-butenyl; aryl groups such as phenyl, 1-naphthyl, 2-naphthyl, 1-anthryl, 2-anthryl, 9-anthryl, 1-phenanthryl, 2-phenanthryl and benzyl; halogen atoms such as F, Cl and Br; alkoxy groups such as methoxy, ethoxy, propoxy and butoxy; and other ones such as hydroxyl, carboxyl, acyl, amino, thio and nitro groups can be exemplified. Further, as a ring system which is formed by binding of R1 and R2, cyclic alkyl groups such as cyclopentane, cyclohexane, cycloheptane, cyclooctane, cyclononane and cyclodecane; aromatic rings such as benzene, naphthalene and anthracene; and heterocycles such as pyridine, pyrrolidine, piperidine, furan, pyran, tetrahydrofuran and tetrahydropyran can be exemplified.

As a carbonyl compound represented in the general formula (IV), specifically aldehydes such as acetaldehyde, propionaldehyde, butylaldehyde, isobutylaldehyde, valeraldehyde, isovaleraldehyde, caproicaldehyde, heptaldehyde, caprylaldehyde, pelargonicaldehyde, capricaldehyde, undecylaldehyde, lauraldehyde, tridecylaldehyde, myristaldehyde, pentadecylaldehyde, palmiticaldehyde, margaricaldehyde, stearicaldehyde, succindialdehyde, acrolein, crotonaldehyde, benzaldehyde, o-tolualdehyde, m-tolualdehyde, p-tolualdehyde, salicylaldehyde, cinnamaldehyde, 1-naphthoaldehyde, 2-naphthoaldehyde and furfural can be exemplified.

Further, as a carbonyl compound represented in the general formula (IV), specifically ketones such as acetone; ethylmethylketone; propylmethylketone; isopropylmethylketone; butylmethylketone; isobutylmethylketone; diethylketone; diisopropylketone; 2-undecanone; methylvinylketone; mesityloxide; fluoroacetone; chloroacetone; 2,4-pentanedione; cyclobutanone; cyclopentanone; cyclohexanone; 2-methylcyclohexanone; cyclodecanone; 2-norbornanone; 2-adamantanone; tetrahydropyrane-4-one; spiro[4,5]-1,4-dioxy-decane-8-one; 1-benzylcarbonylpyperidine-4-one; benzylacetone; 1-indanone; 2-indanone; α-tetralone; β-tetralone; 7-methoxy-2-tetralone; acetophenone; propiophenone; benzylphenone; dibenzylketone; 3,4-dimethylacetophenone; 2-acetonaphthone; and 2-choroloacetophenone can be exemplified.

As a nitroso compound used in a process for producing α-aminooxyketone compounds of the present invention, it may be either of an aliphatic nitroso compound or an aromatic nitroso compound as long as the compound contains a nitroso group. As an aliphatic nitroso compound, an alkyl nitroso compound which may contain substituents can be exemplified and those in which a nitroso group is attached to the tertiary carbon are preferred with specific examples being 2-nitroso-isobutane and 2-nitroso-2-methylpentane. Further, as an aromatic nitroso compound, nitroso benzene which may contain substituents, and 1-nitrosonaphthalene and 2-nitrosonaphthalene which may contain substituents, can be exemplified. As a substituent of aromatic nitroso compound, alkyl groups such as methyl, ethyl, propyl, isopropyl, n-butyl, isobutyl, s-butyl, and t-butyl; alkoxy groups such as methoxy, ethoxy, n-propoxy, isopropoxy, n-butoxy, s-butoxy, isobutoxy, t-butoxy, phenoxy, benzyloxy, andphenethyloxy; and halogen atoms such as F, Cl, Br, and I can be exemplified. As a nitrosobenzene containing substituent group, specifically o-nitrosotoluene; m-nitrosotoluene; p-nitrosotoluene; 3,5-dimethylnitrosobenzene; o-nitrosoethylbenzene; o-nitrosostyrene; o-nitrosoanisole; m-nitrosoanisole; p-nitrosoanisole; o-nitrosophenetol; m-nitrosophenetol; p-nitrosophenetol; o-fluoronitrosobenzene; m-fluoronitrosobenzene; p-fluoronitrosobenzene; o-chrolonitrosobenzene; m-chrolonitrosobenzene; p-chloronitrosobenzene; o-bromonitrosobenzene; m-bromonitrosobenzene; p-bromonitrosobenzene; and the like can be exemplified.

A heterocyclic compound used in the process for producing α-aminooxyketone compounds of the present invention is shown in the general formula (I) wherein X1, X2 and X3 independently represent nitrogen, carbon, oxygen or sulfur; Z represents a substituted or unsubstituted 5- to 10-membered ring, and is a compound in which a 5-membered heterocycle and 5- to 10-membered heterocycle both having nitrogen atom at the respective a position are bound through carbons constituting the heterocycles. The heterocyclic compound is also called an N—H acid-N—H base combined catalyst where the NH functional group at the a position of the heterocycle acts as an acid and the NH functional group at the a position of the cycloalkyl heterocycle acts as a base. As a 5-membered heterocycle (acid ring) of the heterocyclic compound, tetrazole, 1,2,3-triazole, 1,2,4-triazole, pyrazole, pyrazoline, imidazole, indazoline, thiotriazoline and oxatriazoline can be exemplified, and 1H-tetrazole as shown in the genenral formula (II) is especially preferable as a heterocyclic compound. As a 5-10 membered heterocycle (base ring), pyrrolidine, piperidine, hexamethyleneimine, heptamethyleneimine, oxazoline and oxazole can be exemplified; and as a substituent of these heterocycles, alkyl groups such as methyl, ethyl, n-propyl, isopropyl, n-butyl, isobutyl, sec-butyl, and t-butyl, alkoxy groups such as methoxy and ethoxy, phenyl groups, aryl groups such as condensed ring system condensed to heterocycle can be exemplified. Smaller substituents are preferable since a compound with a bulky heterocyclic moiety including substituents would lower the yield.

As the above heterocyclic compound, specifically 5-(2′-pyrrolidinyl)-1H-1,2,3,4-tetrazole; 5-(4H,5H-2′-oxazolyl)-1H-1,2,3,4-tetrazole; 5-(2′-piperidinyl)-1H-1,2,3,4-tetrazole; 5-(benzo[c]-2′-piperidinyl)-1H-1,2,3,4-tetrazole; 5-2′-pyrolidinyl-1H-1,2,3-triazole; 5-2′-pyrolidinyl-1H-1,2,4-triazole; 2-2′-pyrolidinyl-1H-imidazole; 5-2′-pyrolidinyl-1H-imidazole; 5-2′-pyrolidinyl-1H,4H,5H-1,2,3,4-thiotriazoline; 5-2′-pyrolidinyl-4H,5H-1,2,3,4-oxatriazoline; and 5-2′-pyrolidinyl-4H,5H-pyrazoline; can be exemplified. Particularly, 5-(2′-pyrolidinyl)-1H-1,2,3,4-tetrazole as shown in the formura (III) can be preferably exemplified.

The heterocyclic compound can be prepared from natural or synthesized proline. The tetrazole derivative shown in the formula (III) can be prepared by a previous method (Roczniki Chemii Ann. Soc. Chim. Polpnorum, 1971, 45, 967; J. Med. Chm. 1985, 28, 1067). Thus, N-(benzyloxycarbonyl)-L-proline, which is commercially available as a carboxylic acid of proline having nitrogen atom protected by a benzyloxycarbonyl group, is converted to an amide via a reaction with ammonia and dehydrated with phosphoryl chloride to give nitrile. The obtained nitrile is reacted with sodium azide to give a tetrazole, and finally the N-benzyloxycarbonyl group is deprotected with HBr/AcOH or Pd/C, H2 to give a tetrazole derivative shown in the formula (III). The tetrazole derivative can also be obtained according to the previous method from Organic Letters, 2001, Vol. 3, No. 25, 4091-4094; Organic Letters, 2002, Vol. 4 No. 15, 2525-2527; and the like.

A process for producing α-aminooxyketone compounds of the present invention is a method of performing the reaction of carbonyl compound and nitroso compound generally in solvent in the presence of the heterocyclic compound shown in the general formula (I) or preferably in the presence of the tetrazole derivative shown in the formula (III). The amount of nitroso compound can be in a range of 2-4 mol equivalents, preferably 2.5-3.5 mol equivalents for the carbonyl compound, and the amount of catalyst containing the heterocyclic compound shown in the formula (I) can be 1-30 mol %, preferably 2-20 mol % for carbonyl substrate. As a solvent, water; halogenated hydrocarbons such as dichloromethane, chloroform, dichloroethane, and chlorobenzene; aromatic hydrocarbons such as benzene, toluene, xylene; aliphatic hydrocarbons such as cyclohexane, n-hexane, and n-heptane; esters such as ethylacetate; nitrites such as acetonitrile; dimethylsulfoxide; and the like can be exemplified; and among them dimethylsulfoxide and acetonitrile are preferable. The amount of solvent can be 15-30 equivalents but the reaction can be done without solvent.

The reaction temperature can be 0-50° C., preferably 20-30° C. and the reaction can be done at room temperature. The reaction time can be 30 minutes to 3 hours, for example, around 1 hour, and a method stirring in the open air can be exemplified. Strict conditions are not required for a reaction, furthermore, water does not suppress the reaction to proceed, so there is no need for dehydrating the material and catalyst, and the reaction is easy to be controlled. After the reaction is complete, the resulting product was extracted with ethylacetate and the like, and dried and purified by known methods.

In a process for producing α-aminooxyketone compounds of the present invention, carbonyl compounds such as methylisopropylketone and acetophenone, and aldehydes such as water-soluble aldehyde and aldehyde that forms hydrate easily, which could not be used for the materials in conventional methods, can be used as materials. Since there are not many limitations for carbonyl compounds used as a material, the resulting products, α-aminooxyketone compounds, range over a wide scope and have a high chemical yield and optical purity. As an absolute configuration of asymmetric carbons of the resulting product, α-aminooxyketone compound; where aldehyde is used as a material, the resulting product has a R-configuration; where ketone is used as a material, the resulting product has a S-configuration.

Specific examples of the α-aminooxyalketone obtained from the present invention are as follows: (N-isobutylaminooxy)acetaldehyde; [N-(1,1-dimethylbutyl)]aminooxyacetaldehyde; (N-phenylaminooxy)acetaldehyde; 2-(N-isobutylaminooxy)propanal; 2-[N-(1,1-dimethylbutyl)aminooxy]propanal; 2-N-phenylaminooxypropanal; 2-(N-isobutylaminooxy)butanal; 2-[N-(1,1-dimethylbuthyl)aminooxy]butanal; 2-(N-phenylaminooxy)butanal; 2-(N-isobutylaminooxy)-2-methylpropanal; 2-[N-(1,1-dimethylbuthyl)aminooxy]-2-methylpropanal; 2-(N-phenylaminooxy)-2-methylpropanal; 2-(N-isobutylaminooxy)-4-methylbutanal; 2-[N-(1,1-dimethylbutyl)aminooxy]-4-methylbutanal; 2-(N-phenylaminooxy)-4-methylbutanal; 2-(N-isobutylaminooxy)hexanal; 2-[N-(1,1-dimethylbutyl)aminooxy]hexanal; 2-(N-phenylaminooxy)hexanal; 2-(N-isobutylaminooxy)heptanal; 2-[N-(1,1-dimethylbutyl)aminooxy]heptanal; 2-(N-phenylaminooxy)heptanal; 2-(N-isobutylaminooxy)octanal; 2-[N-(1,1-dimethylbutyl)aminooxy]octanal; 2-(N-phenylaminooxy)octanal; 2-(N-isobutylaminooxy)nonanal; 2-[N-(1,1-dimethylbutyl)aminooxy]nonanal; 2-(N-phenylaminooxy)nonanal; 2-(N-isobutylaminooxy)decanal; 2-[N-(1,1-dimethylbutyl)aminooxy]decanal; 2-(N-phenylaminooxy)decanal; 2-(N-isobutylaminooxy)undecanal; 2-[N-(1,1-dimethylbutyl)aminooxy]undecanal; 2-(N-phenylaminooxy)propanal; 2-(N-isobutylaminooxy)dodecanal; 2-[N-(1,1-dimethylbutyl)aminooxy]dodecanal; 2-(N-phenylaminooxy)dodecanal; 2-(N-isobutylaminooxy)tridecanal; 2-[N-(1,1-dimethylbutyl)aminooxy]tridecanal; and 2-(N-phenylaminooxy)tridecanal.

Further specific examples of the α-aminooxyketone compound obtained from the present invention are as follows: 2,3-bis(N-isobutylaminooxy)butanedial; 2,3-bis[N-(1,1-dimethylbutyl)aminooxy]butanedial; 2,3-bis[N-phenylaminooxy]butanedial; 2-N-isobutylaminooxy-2-propenal; 2-N-(1,1-dimethylbutyl)aminooxy-2-propenal; 2-N-phenylaminooxy-2-propenal; 2-N-isobutylaminooxy-2-butenal; 2-N-(1,1-dimethylbutyl)aminooxy-2-butenal; 2-N-phenylaminooxy-2-butenal; 3-phenyl-2-N-isobutylaminooxy-2-propenal; 3-phenyl-2-N-(1,1-dimethylbutyl)aminooxy-2-propenal; and 3-phenyl-2-N-phenylaminooxy-2-propenal.

Furthermore, examples of the α-aminooxyketone obtained from the present invention are as follows: (N-isobutylaminooxy)acetone; [N-(1,1-dimethylbutyl)aminooxy]acetone; (N-phenylaminooxy)acetone; 3-(N-isobutylaminooxy)butane-2-one; 3-[N-(1,1-dimethylbutyl)aminooxy]butane-2-one; 3-(N-phenylaminooxy)butane-2-one; 3-(N-isobutylaminooxy)pentane-2-one; 3-[N-(1,1-dimethylbutyl)aminooxy]pentane-2-one; 3-(N-phenylaminooxy)pentane-2-one; 3-(N-isobutylaminooxy)-4-methylbutane-2-one; 3-[N-(1,1-dimethylbutyl)aminooxy]-4-methylbutane-2-one; 3-(N-phenylaminooxy)-4-methylbutane-2-one; 3-(N-isobutylaminooxy)hexane-2-one; 3-[N-(1,1-dimethylbutyl)aminooxy]hexane-2-one; 3-(N-phenylaminooxy)hexane-2-one; 3-(N-isobutylaminooxy)-4-methylpentane-2-one; 3-[N-(1,1-dimethylbutyl)aminooxy]-4-methylpentane-2-one; 3-(N-phenylaminooxy)-4-methylpentane-2-one; 2-(N-isobutylaminooxy)pentane-3-one; 2-[N-(1,1-dimethylbutyl)aminooxy]pentane-3-one; 2-(N-phenylaminooxy)pentane-3-one; 2-(N-isobutylaminooxy)-2,4-dimethylpentane-3-one; 2-[N-(1,1-dimethylbutyl)aminooxy]-2,4-dimethylpentane-3-one; 2-(N-phenylaminooxy)-2,4-dimethylpentane-3-one; 3-(N-isobutylaminooxy)undecane-2-one; 3-[N-(1,1-dimethylbutyl)aminooxy]undecane-2-one; and 3-(N-phenylaminooxy)undecane-2-one.

Further examples of the α-aminooxyketone compound obtained from the present invention are as follows: 3-N-isobutylaminooxy-3-butene-2-one; 3-N-(1,1-dimethylbutyl)aminooxy-3-butene-2-one; 3-N-phenylaminooxy-3-butene-2-one; 3-N-isobutylaminooxy-4-methyl-3-pentene-2-one; 3-N-(1,1-dimethylbutyl)aminooxy-4-methyl-3-pentene-2-one; 3-N-phenylaminooxy-4-methyl-3-pentene-2-one; 1-fluoro-1-(N-isobutylaminooxy)acetone; 1-fluoro-1-[N-(1,1-dimethylbutyl)aminooxy]acetone; 1-fluoro-1-(N-phenylaminooxy)acetone; 1-chloro-1-(N-isobutylaminooxy)acetone; 1-chloro-1-[N-(1,1-dimethylbutyl)aminooxy]acetone; 1-chloro-1-(N-phenylaminooxy)acetone; 3-(N-isobutylaminooxy)-2,4-pentanedione; 3-[N-(1,1-dimethylbutyl)aminooxy]-2,4-pentanedione; 3-(N-phenylaminooxy)-2,4-pentanedione; 2-(N-isobutylaminooxy)cyclobutanone; 2-[N-(1,1-dimethylbutyl)aminooxy]cyclobutanone; 2-(N-phenylaminooxy)cyclobutanone; 2-(N-isobutylaminooxy)cyclopentanone; 2-[N-(1,1-dimethylbutyl)aminooxy]cyclopentanone; 2-(N-phenylaminooxy)cyclopentanone; 2-(N-isobutylaminooxy)cyclohexanone; 2-[N-(1,1-dimethylbutyl)aminooxy]cyclohexanone; 2-(N-phenylaminooxy)cyclohexanone; 2-(N-isobutylaminooxy)-2-methylcyclohexanone; 2-[N-(1,1-dimethylbutyl)aminooxy]-2-methylcyclohexanone; 2-(N-phenylaminooxy)-2-methylcyclohexanone; 2-(N-isobutylaminooxy)cyclodecanone; 2-[N-(1,1-dimethylbutyl)aminooxy]cyclodecanone; 2-(N-phenylaminooxy)cyclodecanone; 1-(N-isobutylaminooxy)-2-norbornanone; 1-[N-(1,1-dimethylbutyl)aminooxy]-2-norbornanone; 1-(N-phenylaminooxy)-2-norbornanone; 1-(N-isobutylaminooxy)-2-adamantanone; 1-[N-(1,1-dimethylbutyl)aminooxy]-2-adamantanone; and 1-(N-phenylaminooxy)-2-adamantanone.

And more examples are as follows: 2-(N-isobutylaminooxy)-4-tetrahydropyranone; 2-[N-(1,1-dimethylbutyl)aminooxy]-4-tetrahydropyranone; 2-(N-phenylaminooxy)-4-tetrahydropyranone; 7-(N-isobutylaminooxy)-spiro[4.5]-1,4-dioxy-decane-8-one; 7-[N-(1,1-dimethylbutyl)aminooxyl-spiro[4.5]-1,4-dioxy-decane-8-one; 7-(N-phenylaminooxy)-spiro[4.5]-1,4-dioxy-decane-8-one; 3-(N-isobutylaminooxy)-1-benzylcarbonylpiperidine-4-one; 3-[N-(1,1-dimethylbutyl)aminooxy]-1-benzylcarbonylpiperidine-4-one; 3-(N-phenylaminooxy)-1-benzylcarbonylpiperidine-4-one; 3-(N-isobutylaminooxy)-4-phenylbutane-2-one; 3-[N-(1,1-dimethylbutyl)aminooxy-4-phenylbutane-2-one; 3-(N-phenylaminooxy)-4-phenylbutane-2-one; 2-(N-isobutylaminooxy)-1-indanone; 2-[N-(1,1-dimethylbutyl)aminooxy]-1-indanone; 2-(N-phenylaminooxy)-1-indanone; 1-(N-isobutylaminooxy)-2-indanone; 1-[N-(1,1-dimethylbutyl)aminooxy-2-indanone; 1-(N-phenylaminooxy)-2-indanone; 2-(N-isobutylaminooxy)-1-ketotetrahydronaphthalene; 2-[N-(1,1-dimethylbutyl)aminooxy-1-ketotetrahydronaphthalene; 2-(N-phenylaminooxy)-1-ketotetrahydronaphthalene; 1-(N-isobutylaminooxy)-2-ketotetrahydronaphthalene; 1-[N-(1,1-dimethylbutyl)aminooxy]-2-ketotetrahydronaphthalene; 1-(N-phenylaminooxy)-2-ketotetrahydronaphthalene; 1-(N-isobutylaminooxy)-7-methoxy-2-ketotetrahydronaphthalene; 1-[N-(1,1-dimethylbutyl)aminooxy-7-methoxy-2-ketotetrahydronaphthalene; 1-(N-phenylaminooxy)-7-methoxy-2-ketotetrahydronaphthalene; 2′-(N-isobutylaminooxy)-1-acetophenone; 2′-[N-(1,1-dimethylbutyl)aminooxy]-1′-acetophenone; 2′-(N-phenylaminooxy)-1′-acetophenone; 2′-(N-isobutylaminooxy)-1′-propiophenone; 2′-[N-(1,1-dimethylbutyl)aminooxy]-1′-propiophenone; 2′-(N-phenylaminooxy)-1′-propiophenone; 2-(N-isobutylaminooxy)-1,2-bisphenylethane-1-one; 2-[N-(1,1-dimethylbutyl)aminooxy]-1,2-bisphenylethane-1-one; 2-(N-phenylaminooxy)-1,2-bisphenylethane-1-one; 1-(N-isobutylaminooxy)-1,3-bisphenylpropane-2-one; 1-[N-(1,1-dimethylbutyl)aminooxy]-1,3-bisphenylpropane-2-one; 1-(N-phenylaminooxy)-1,3-bisphenylpropane-2-one; 6-(N-isobutylaminooxy)-3,4-dimethylacetophenone; 6-[N-(1,1-dimethylbutyl)aminooxy]-3,4-dimethylacetophenone; 6-(N-phenylaminooxy)-3,4-dimethylacetophenone; 3′-(N-isobutylaminooxy)-2′-acetonaphthone, 3′-[N-(1,1-dimethylbutyl)aminooxy]-2′-acetonaphthone; 3′-(N-phenylaminooxy)-2′-acetonaphthone; 3′-(N-isobutylaminooxy)-2′-chloroacetonaphthone; 3′-[N-(1,1-dimethylbutyl)aminooxy]-2′-acetonaphtone; and 3′-(N-phenylaminooxy)-2′-acetonaphtone.

Further, the process for producing α-hydroxyketone compounds of the present invention is a process using an α-aminooxyketone compound obtained from the above mentioned process for producing α-aminooxyketone compounds of the present invention in a solvent with the use of CuSO₄ as a catalyst, and to which known methods can be applied to convert aminooxyketone compound to hydroxyketone compound. Alcohols like methanol and ethanol can be exemplified as a solvent to use. The reaction temperature can be about 0-25° C., and the reaction time can be about 3-10 hours.

The compounds of the invention may be useful for treating or preventing a variety of cancers, including, but not limited to, leukemias, including but not limited to acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, Lymphomas including but not limited to Hodgkin's disease, non-Hodgkin's disease, Multiple myeloma, Waldenstrom's macroglobulinemia, Heavy chain disease, Solid tumors including but not limited to sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma.

In another embodiment, the compounds of the present invention may be useful in preventing or treating cardiovascular diseases, such as, but not limited to, hypertension, heart failure, pulmonary hypertension and renal diseases. For example, bosentan, an endothelin receptor antagonist, has received approval by the Food and Drug Administration (FDA) for use in pulmonary artery hypertension (see, e.g., Vatter et al., Methods Find Exp Clin Pharmacol. May 2004;26(4):277-86). The compounds of the present invention, or a derivative thereof, may be used as an endothelin receptor antagonist (see, e.g., Niiyama et al., Bioorg Med Chem. November 2002;10(11):3437-44).

In another embodiment, the compounds of the present invention may be useful in neutralizing toxins, such as, but not limited to, SLTs, verotoxins, cholera toxin, clostridium difficile toxins A and B, bacterial pili from enteropathogenic E. coli (EPEC) and enterotoxigenic E. coli (ETEC).

Due to their activity, the compounds of the invention are advantageously useful in veterinary and human medicine.

When administered to a patient, a compound of the invention is preferably administered as component of a composition that optionally comprises a pharmaceutically acceptable vehicle the present compositions, which comprise a compound of the invention, are preferably administered orally. The compositions of the invention may also be administered by any other convenient route, for example, by infusion or bolus injection, by absorption through epithelial or mucocutaneous linings (e.g., oral mucosa, rectal, and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer the compounds of the invention.

In certain embodiments, the present compositions may comprise one or more compounds of the invention.

Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The mode of administration is left to the discretion of the practitioner. In most instances, administration will result in the release of a compound of the invention into the bloodstream.

In specific embodiments, it maybe desirable to a compound of the invention locally. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

In certain embodiments, it may be desirable to introduce a compound of the invention into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the compounds of the invention can be delivered in a vesicle, in particular a liposome (see Langer, 1990. Science 249:1527-1533; Treat et al, in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid).

In yet another embodiment, the compounds of the invention can be delivered in a controlled release system (see, e.g., Goodson, in Medical Applications of Controlled Release, supra, vol. 2, pp. 115-138 (1984)). Other controlled-release systems discussed in the review by Langer, 1990, Science 249:1527-1533) may be used. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of a target of a compound of the invention, thus requiring only a fraction of the systemic dose.

The present compositions can optionally comprise a suitable amount of a pharmaceutically acceptable vehicle so as to provide the form for proper administration to the patient.

In a specific embodiment, the term “pharmaceutically acceptable” means approved by a regulatory agency of the Federal or a state government or listed in the U.S. Pharmacopeia or other generally recognized pharmacopeia for use in animals, mammals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening. lubricating and coloring agents may be used. When administered to a patient, the pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, oranyother form suitable for use. In one embodiment, the pharmaceutically acceptable vehicle is a capsule (see e.g., U.S. Pat. No. 5,698,155). Other examples of suitable pharmaceutical vehicles are described in Remington's Pharmaceutical Sciences, Alfonso R. Gennaro ed., Mack Publishing Co. Easton, Pa., 19th ed., 1995, pp. 1447 to 1676, incorporated herein by reference.

In a preferred embodiment, the compounds of the invention are formulated in accordance with routine procedures as a pharmaceutical composition adapted for oral administration to human beings. Compositions for oral delivery may be in the form of tablets, lozenges, aqueous or oily suspensions, granules, powders, emulsions, capsules, syrups, or elixirs, for example. Orally administered compositions may contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compositions. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. Typically, compositions for intravenous administration comprise sterile isotonic aqueous buffer. Where necessary, the compositions may also include a solubilizing agent.

In another embodiment, the compounds of the invention can be formulated for intravenous administration. Compositions for intravenous administration may optionally include a local anesthetic such as lignocaine to lessen pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compounds of the invention are to be administered by infusion, they can be dispensed, for example, with an infusion bottle containing sterile pharmaceutical grade water or saline. Where the compounds of the invention are administered by injection, an ampoule of sterile water for injection or saline can be provided so that the ingredients may be mixed prior to administration.

The amount of a compound of the invention that will be effective in the treatment of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed will also depend on the route of administration, and the seriousness of the disease or disorder, and should be decided according to the judgment of the practitioner and each patient's circumstances. However, suitable dosage ranges for oral administration are generally about 0.001 milligram to about 200 milligrams of a compound of the invention or a pharmaceutically acceptable salt thereof per kilogram body weight per day. In specific preferred embodiments of the invention, the oral dose is about 0.01 milligram to about 100 milligrams per kilogram body weight per day, more preferably about 0.1 milligram to about 75 milligrams per kilogram body weight per day, more preferably about 0.5 milligram to 5 milligrams per kilogram body weight per day. The dosage amounts described herein refer to total amounts administered; that is, if more than one compound of the invention is administered, or if a compound of the invention is administered with a therapeutic agent, then the preferred dosages correspond to the total amount administered. Oral compositions preferably contain about 10% to about 95% active ingredient by weight.

Suitable dosage ranges for intravenous (i.v.) administration are about 0.01 milligram to about 100 milligrams per kilogram body weight per day, about 0.1 milligram to about 35 milligrams per kilogram body weight per day, and about 1 milligram to about 10 milligrams per kilogram body weight per day. Suitable dosage ranges for intranasal administration are generally about 0.01 pg/kg body weight per day to about 1 mg/kg body weight per day. Suppositories generally contain about 0.01 milligram to about 50 milligrams of a compound of the invention per kilogram body weight per day and comprise active ingredient in the range of about 0.5% to about 10% by weight.

Recommended dosages for intradermal, intramuscular, intraperitoneal, subcutaneous, epidural, sublingual, intracerebral, intravaginal, transdermal administration or administration by inhalation are in the range of about 0.001 milligram to about 200 milligrams per kilogram of body weight per day, Suitable doses for topical administration are in the range of about 0.001 milligram to about 1 milligram, depending on the area of administration. Effective doses may be extrapolated from dose-response curves derived from in vitro or animal model test systems. Such animal models and systems are well known in the art.

The invention also provides pharmaceutical packs or kits comprising one or more vessels containing one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound of the invention. In another embodiment, the kit comprises a therapeutic agent and a compound of the invention.

The compounds of the invention are preferably assayed in vitro and in vivo, for the desired therapeutic or prophylactic activity, prior to use in humans. For example, in vitro assays can be used to determine whether it is preferable to administer a compound of the invention alone or in combination with another compound of the invention and/or a therapeutic agent. Animal model systems can be used to demonstrate safety and efficacy.

Other methods will be known to the skilled artisan and are within the scope of the invention.

In certain embodiments of the present invention, a compound of the invention can be used in combination therapy with at least one other therapeutic agent. The compound of the invention and the therapeutic agent can act additively or, more preferably, synergistically. In a preferred embodiment, a composition comprising a compound of the invention is administered concurrently with the administration of another therapeutic agent, which can be part of the same composition as or in a different composition from that comprising the compound of the invention. In another embodiment, a composition comprising a compound of the invention is administered prior or subsequent to administration of another therapeutic agent. As many of the disorders for which the compounds of the invention are useful in treating are chronic, in one embodiment combination therapy involves alternating between administering a composition comprising a compound of the invention and a composition comprising another therapeutic agent, e.g., to minimize the toxicity associated with a particular drug. The duration of administration of the compound of the invention or therapeutic agent can be, e.g., one month, three months, six months, a year, or for more extended periods. In certain embodiments, when a compound of the invention is administered concurrently with another therapeutic agent that potentially produces adverse side effects including, but not limited to, toxicity, the therapeutic agent can advantageously be administered at a dose that falls below the threshold at which the adverse side is elicited.

The therapeutic agent can be an anti-cancer agent. Useful anti-cancer agents include, but are not limited to, methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel, .gamma.-radiation, alkylating agents including nitrogen mustard such as cyclophosphamide, Ifosfamide, trofosfamide, Chlorambucil, nitrosoureas such as carmustine (BCNU), and Lomustine (CCNU), alkylsulphonates such as busulfan, and Treosulfan, triazenes such as Dacarbazine, platinum containing compounds such as Cisplatin and carboplatin, plant alkaloids including vinca alkaloids, vincristine, Vinblastine, Vindesine, and Vinorelbine, taxoids including paclitaxel, and Docetaxol, DNA topoisomerase inhibitors including Epipodophyllins such as etoposide, Teniposide, Topotecan, 9-aminocamptothecin, campto irinotecan, and crisnatol, mytomycins such as mytomycin C, and Mytomycin C, anti-metabolites, including anti-folates such as DHFR inhibitors, methotrexate and Trimetrexate, IMP dehydrogenase inhibitors including mycophenolic acid, Tiazofurin, Ribavirin, EICAR, Ribonuclotide reductase Inhibitors such as hydroxyurea, deferoxamine, pyrimidine analogs including uracil analogs 5-Fluorouracil, Floxuridine, Doxifluridine, and Ratitrexed, cytosine analogs such as cytarabine (ara C), cytosine arabinoside, and fludarabine, purine analogs such as mercaptopurine, thioguanine, hormonal therapies including receptor antagonists, the anti-estrogens Tamoxifen, Raloxifene and megestrol, LHRH agonists such as goscrclin, and Leuprolide acetate, anti-androgens such as flutamide, and bicalutamide, retinoids/deltoids, Vitamin D3 analogs including EB 1089, CB 1093, and KH 1060, photodyamic therapies including vertoporfin (BPD-MA), Phthalocyanine, photosensitizer Pc4, Demethoxy-hypocrellin A, (2BA-2-DMHA), cytokines including Interferon-.alpha., Interferon-.gamma., tumor necrosis factor, as well as other compounds having anti-tumor activity including Isoprenylation inhibitors such as Lovastatin, Dopaminergic neurotoxins such as 1-methyl-4-phenylpyridinium ion, Cell cycle inhibitors such as staurosporine, Actinomycins such as Actinomycin D and Dactinomycin, Bleomycins such as bleomycin A2, Bleomycin B2, and Peplomycin, anthracyclines such as daunorubicin, Doxorubicin (adriamycin), Idarubicin, Epirubicin, Pirarubicin, Zorubicin, and Mitoxantrone, MDR inhibitors including verapamil, and Ca2+ ATPase inhibitors such as thapsigargin.

The therapeutic agent can be an antiviral agent. Useful antiviral agents include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons.

The therapeutic agent can be an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, diclofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; and other anti-inflammatory agents including, but not limited to, colchicine, allopurinol, probenecid, sulfinpyrazone and benzbromarone.

The invention will now be further described by way of the following non-limiting examples.

### EXAMPLES

### Example 1

Pyrrolidine-2-tetrazole 1 was prepared from proline. One mL dimethyl sulfoxide (DMSO) solution at room temperature (25-30° C.) in which 5 mol % Pyrrolidine-2-tetrazole and 1.5 mmol (3 equivalents) cyclohexanone was dissolved and was added dropwise 1 mL DMSO solution with 0.5 mmol (1 equivalent) nitrosobenzene for 1 hour. The mixture was stirred at room temperature and allowed to react for 1 hour. The nitrosobenzene was completely consumed, as determined by TLC (hexane/ethyl acetate=3/1). The desired product (4), 2-(N-phenylaminooxy)-1-cyclohexanone was obtained. It was 94% chemical yield and >99% ee optical purity. The results are shown in Table 1. In Table 1, chemical yield ratio (4/6) shows the yield ratio of isolated isomer, optical purity (ee %) of the desired product shows the measurement of HPLC, and absolute configuration (R/S) of asymmetric carbon of the desired products shows the yield of diol converted from the desired products. Even where the amount of catalyst 1 to be used was changed to 3 mol % and 2 mol %, the reactions progressed. The chemical yield of the desired products was 72% and 50% respectively, but the optical purities were >99% ee.

### Comparative Example 1

Reaction was performed in the same manner as Example 1 with the exception of using proline as a catalyst. The chemical yield of the resulting desired product stayed at 35%, and the production of double additional product (5a) of about 2% of 2,6-bis(N-phenylaminooxy)-1-cyclohexanone was identified.

### Example 2

Reaction was performed in the same manner as Example 1 with the exception of using tetrahydropyrane-4-one as a carbonyl compound. Chemical yield and optical purity of the desired products are shown in Table 1.

### Example 3

Reaction was performed in the same manner as Example 1 with the exception of using spiro[4.5]-1,4-dioxy-decane-8-one as a carbonyl compound. Chemical yield and optical purity of the desired products are shown in Table 1.

### Example 4

Reaction was performed in the same manner as Example 1 with the exception of using 1-benzylcarbonylpiperidine-4-one as a carbonyl compound. Chemical yield and optical purity of the desired products are shown in Table 1.

### Example 5

Reaction was performed in the same manner as Example 1 with the exception of using methylethylketone as a carbonyl compound and the amount of 20 mol % catalyst. Chemical yield and optical purity of the desired products are shown in Table 1.

### Example 6

Reaction was performed in the same manner as Example 1 with the exception of using phenylpropionaldehyde as a carbonyl compound, acetonitrile as a solvent, and the amount of 10 mol % catalyst. Chemical yield and optical purity of the desired products are shown in Table 1. In the Table, chemical yield of the desired products shows the yield of primary alcohol obtained by reduction of the desired products.

### Example 7

Reaction was performed in the same manner as Example 6 with the exception of using isobutyraldehyde as a carbonyl compound and the amount of 20 mol % catalyst. Chemical yield and optical purity of the desired products are shown in Table 1.

### Example 8

Reaction was performed in the same manner as Example 6 with the exception of using caproicaldehyde as a carbonyl compound. Chemical yield and optical purity of the desired products are shown in Table 1.

TABLE 1

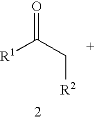

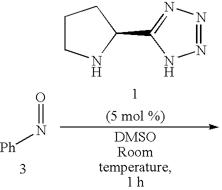

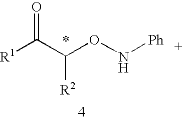

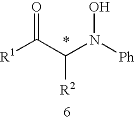

| | | | | | | Optical | |
|---|---|---|---|---|---|---|---|
| | | | | Yield | Yield | purity4 | R/S |
| Example 2 | | 1 (mol %) | Solvent | (%) | ratio4/6 | (ee %) | configuration |
| 1 | 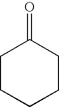 | 5 | DMSO | 94 | >99/— | >99 | S |
| 2 | 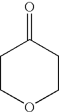 | 5 | DMSO | 87 | >99/— | >99 | S |
| 3 | 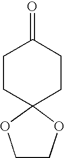 | 5 | DMSO | 97 | >99/— | 99 | S |
| 4 | 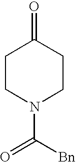 | 5 | DMSO | 95 | >99/— | >99 | S |
| 5 | 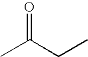 | 20 | DMSO | 75 | 72/28 | >99 | S |
| 6 | 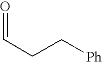 | 10 | MeCN | 67 | >99/— | 98 | R |
| 7 | 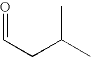 | 20 | MeCN | 65 | >99/— | 98 | R |
| 8 | 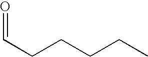 | 10 | MeCN | 69 | >99/— | 98 | R |

### Example 9

DMSO solution containing 1 mmol of the resulting products from Example 1 was directly cooled to 0° C., added 47.9 mg (0.3 mmol) CuSO₄ and 3.0 mL methanol, and stirred at 0° C. for 10 hours. The reaction mixture was added 20 mL cooled saline solution as aftertreatment, and extracted 3 times with 10 mL ethyl acetate. The extracted organic phase was all collected and washed with saline solution, filtered after drying with Na₂SO₄, and distilled the resulting filtrate with evaporator under reduced pressure. After the residue was purified with silica gel chromatography (developing solvent: ethyl acetate/hexane), corresponding α-hydroxyketone was obtained. The yield was 90%, and optical yield was 99% or more.

The invention is further described by the following numbered paragraphs:

1. A process for producing an α-aminooxyketone compound wherein a carbonyl compound is reacted with a nitroso compound by using a catalyst containing a heterocyclic compound shown in the general formula (I) (wherein: X1, X2 and X3 independently represent nitrogen, carbon, oxygen or sulfur; Z represents a substituted or unsubstituted 5- to 10-membered ring).

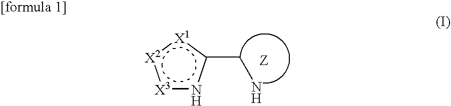

2. The process for producing an α-aminooxyketone compound according to paragraph 1, wherein the heterocyclic compound is a tetrazole derivative shown in the general formula (II) (wherein Z represents a substituted or unsubstituted 5- to 10-membered ring).

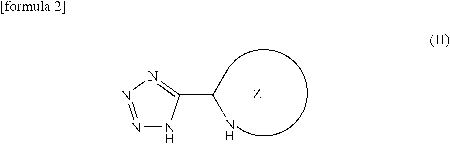

3. The process for producing an α-aminooxyketone compound according to paragraph 2, wherein the tetrazole derivative is a compound shown in the general formula (III).

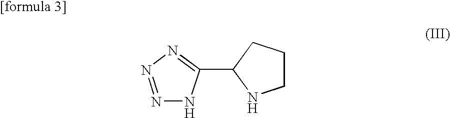

4. The process for producing an α-aminooxyketone compound according to any one of paragraphs 1-3, wherein the nitroso compound is nitrosobenzene.

5. The process for producing an α-aminooxyketone compound according to any one of paragraphs 1-4, wherein the carbonyl compound is a compound shown in the general formula (IV) (wherein R1 and R2 independently represent hydrogen, or a substituted or unsubstituted alkyl group, a substituted or unsubstituted alkenyl group, a substituted or unsubstituted alkoxy group, a substituted or unsubstituted amide group, or a substituted or unsubstituted aryl group; and R1 and R2 may bind to each other to form a ring).

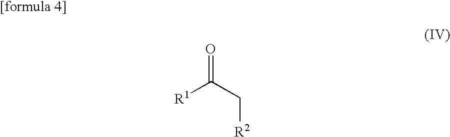

6. The process for producing an α-aminooxyketone compound according to paragraphs 5, wherein the α-aminooxyketone compound contains R asymmetric carbon, where R1 represents hydrogen in the general formula (IV).

7. The process for producing an α-aminooxyketone compound according to paragraphs 5, wherein the α-aminooxyketone compound contains S asymmetric carbon; where R1 represents a substituted or unsubstituted alkyl group, a substituted or unsubstituted alkenyl group, a substituted or unsubstituted alkoxy group, a substituted or unsubstituted amide group, or a substituted or unsubstituted aryl group, and either one of these groups bind to R2 to form a ring in the general formula (IV).

8. The process for producing an α-aminooxyketone compound according to any one of paragraphs 1-7, wherein a solvent containing dimethyl sulfoxide or methyl nitrile, or both of them is used.

9. The process for producing an α-aminooxyketone compound according to any one of paragraphs 1-8, wherein reaction is performed at room temperature (20-30° C.).

10. A process for producing an α-hydroxyketone compound wherein an α-aminooxyketone compound obtained from the process according to any one of paragraphs 1-9 is reacted in a solvent by using CuSO₄ as a catalyst.

11. A catalyst for producing an α-aminooxyketone compound by which a carbonyl compound is reacted with a nitroso compound, wherein the catalyst contains a heterocyclic compound shown in the general formula (I) (wherein: X1, X2 and X3 independently represent nitrogen, carbon, oxygen or sulfur; Z represents a substituted or unsubstituted 5- to 10-membered ring).

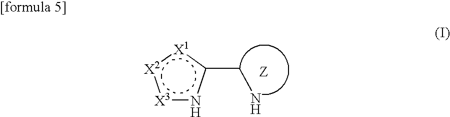

12. The catalyst for producing an α-aminooxyketone compound according to paragraph 11, wherein the heterocyclic compound is a tetrazole derivative shown in the general formula (II) (wherein Z represents a substituted or unsubstituted 5- to 10-membered ring).

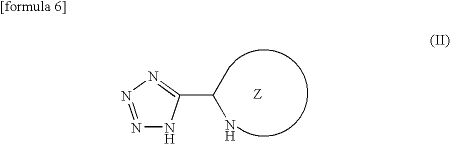

13. The catalyst for producing an α-aminooxyketone compound according to paragraph 12, wherein the tetrazole derivative is a compound shown in the general formula (III).

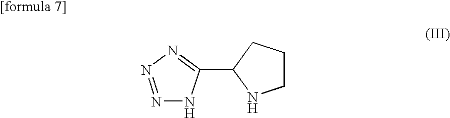

14. The catalyst for producing an α-aminooxyketone compound according to any one of paragraphs 11-13, wherein the nitroso compound is nitrosobenzene.

15. The catalyst for producing an α-aminooxyketone compound according to any one of paragraphs 11-14, wherein the carbonyl compound is a compound shown in the general formula (IV) (wherein R1 and R2 independently represent hydrogen, or a substituted or unsubstituted alkyl group, a substituted or unsubstituted alkenyl group, a substituted or unsubstituted alkoxy group, a substituted or unsubstituted amide group, or a substituted or unsubstituted aryl group; and R1 and R2 may bind to each other to form a ring).

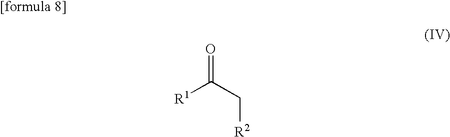

16. The catalyst for producing an α-aminooxyketone compound according to paragraph 15, wherein the α-aminooxyketone compound contains R asymmetric carbon, where R1 represents hydrogen in the general formula (IV).

17. The catalyst for producing an α-aminooxyketone compound according to paragraph 15, wherein the α-aminooxyketone compound contains S asymmetric carbon, where R1 represents a substituted or unsubstituted alkyl group, a substituted or unsubstituted alkenyl group, a substituted or unsubstituted alkoxy group, a substituted or unsubstituted amide group, or a substituted or unsubstituted aryl group, or where either one of these groups bind to R2 to form a ring in the general formula (IV).

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A process for producing an α-aminooxyketone compound wherein a carbonyl compound is reacted with a nitroso compound by using a catalyst containing a heterocyclic compound shown in the general formula (I), wherein: X1, X2 and X3 independently represent nitrogen, carbon, oxygen or sulfur; Z represents a substituted or unsubstituted 5- to 10-membered ring.

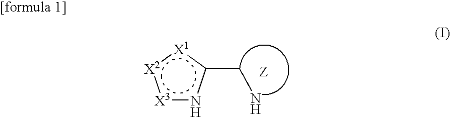

2. The process for producing an α-aminooxyketone compound according to claim 1, wherein the heterocyclic compound is a tetrazole derivative shown in the general formula (II), wherein Z represents a substituted or unsubstituted 5- to 10-membered ring.

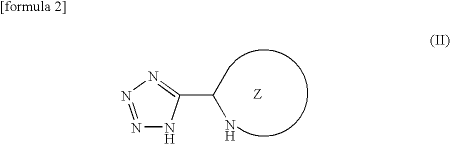

3. The process for producing an α-aminooxyketone compound according to claim 2, wherein the tetrazole derivative is a compound shown in the general formula (III).

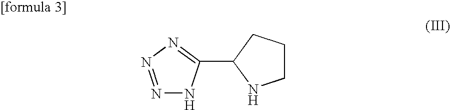

4. The process for producing an α-aminooxyketone compound according to claim 1, wherein the nitroso compound is nitrosobenzene.

5. The process for producing an α-aminooxyketone compound according to claim 1, wherein the carbonyl compound is a compound shown in the general formula (IV), wherein R1 and R2 independently represent hydrogen, or a substituted or unsubstituted alkyl group, a substituted or unsubstituted alkenyl group, a substituted or unsubstituted alkoxy group, a substituted or unsubstituted amide group, or a substituted or unsubstituted aryl group; and R1 and R2 may bind to each other to form a ring.

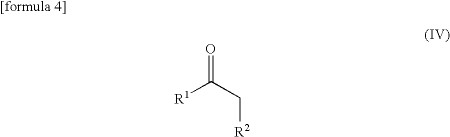

6. The process for producing an α-aminooxyketone compound according to claim 5, wherein the α-aminooxyketone compound contains R asymmetric carbon, where R1 represents hydrogen in the general formula (IV).

7. The process for producing an α-aminooxyketone compound according to claim 5, wherein the α-aminooxyketone compound contains S asymmetric carbon; where R1 represents a substituted or unsubstituted alkyl group, a substituted or unsubstituted alkenyl group, a substituted or unsubstituted alkoxy group, a substituted or unsubstituted amide group, or a substituted or unsubstituted aryl group, and either one of these groups bind to R2 to form a ring in the general formula (IV).

8. The process for producing an α-aminooxyketone compound according to claim 1, wherein a solvent containing dimethyl sulfoxide or methyl nitrile, or both of them is used.

9. The process for producing an α-aminooxyketone compound according to claim 1, wherein reaction is performed at room temperature (20-30° C.).

10. A process for producing an α-hydroxyketone compound wherein an α-aminooxyketone compound obtained from the process according to claim 1 is reacted in a solvent by using CuSO₄ as a catalyst.

11. A catalyst for producing an α-aminooxyketone compound by which a carbonyl compound is reacted with a nitroso compound, wherein the catalyst contains a heterocyclic compound shown in the general formula (I), wherein: X1, X2 and X3 independently represent nitrogen, carbon, oxygen or sulfur; z represents a substituted or unsubstituted 5- to 10-membered ring.

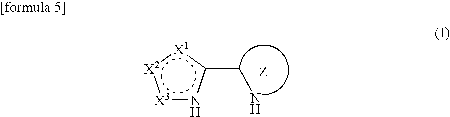

12. The catalyst for producing an α-aminooxyketone compound according to claim 11, wherein the heterocyclic compound is a tetrazole derivative shown in the general formula (II), wherein Z represents a substituted or unsubstituted 5- to 10-membered ring.

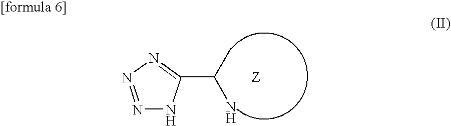

13. The catalyst for producing an α-aminooxyketone compound according to claim 12, wherein the tetrazole derivative is a compound shown in the general formula (III).

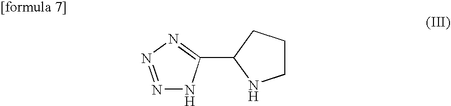

14. The catalyst for producing an α-aminooxyketone compound according to claim 11, wherein the nitroso compound is nitrosobenzene.

15. The catalyst for producing an α-aminooxyketone compound according to claim 11, wherein the carbonyl compound is a compound shown in the general formula (IV), wherein R1 and R2 independently represent hydrogen, or a substituted or unsubstituted alkyl group, a substituted or unsubstituted alkenyl group, a substituted or unsubstituted alkoxy group, a substituted or unsubstituted amide group, or a substituted or unsubstituted aryl group; and R1 and R2 may bind to each other to form a ring.

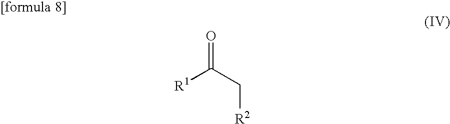

16. The catalyst for producing an α-aminooxyketone compound according to claim 15, wherein the α-aminooxyketone compound contains R asymmetric carbon, where R1 represents hydrogen in the general formula (IV).

17. The catalyst for producing an α-aminooxyketone compound according to claim 15, wherein the α-aminooxyketone compound contains S asymmetric carbon, where R1 represents a substituted or unsubstituted alkyl group, a substituted or unsubstituted alkenyl group, a substituted or unsubstituted alkoxy group, a substituted or unsubstituted amide group, or a substituted or unsubstituted aryl group, or where either one of these groups bind to R2 to form a ring in the general formula (IV).
